(19) United States
(12) Patent Application Publication
(10) Pub. No.: US 2025/0041186 A1
(43) Pub. Date: Feb. 6, 2025

(54) PERSONAL CLEANSING COMPOSITION FREE OF ALKYL SULFATE OR ALKYL ETHER SULFATE TYPE OF SURFACTANTS

(71) Applicant: The Procter & Gamble Company, Cincinnati, OH (US)

(72) Inventors: Lesheng Zhang, Beijing (CN); Andrei Sergeevich Bureiko, Cincinnati, OH (US); Aijun Zhang, Beijing (CN)

(21) Appl. No.: 18/782,827

(22) Filed: Jul. 24, 2024

(30) Foreign Application Priority Data
Aug. 3, 2023 (WO) PCT/CN2023/110907

(51) Int. Cl. A61K 8/46; A61Q 5/02

(52) U.S. Cl. CPC A61K 8/466; A61Q 5/02; A61K 2800/20; A61K 2800/596

(57) ABSTRACT

Personal cleansing composition having a surfactant system. The surfactant system includes from about 1.75% to about 15% of an acyl isethionate surfactant by weight of the composition with a specific chain length distribution in the acyl isethionate surfactant. The pH has from about 5.5 to about 7. The composition is substantially free of alkyl sulfate and alkyl ether sulfate type of surfactants.

### FIELD OF THE INVENTION

The present application generally relates to personal cleansing compositions with an improved rheology, their methods and their uses. The personal cleansing compositions comprise a surfactant system, wherein the surfactant system comprises an acyl isethionate surfactant, with a specific chain length distribution and when the personal cleansing composition is substantially free of alkyl sulfate or alkyl ether sulfate type of surfactants.

### BACKGROUND OF THE INVENTION

Personal cleansing compositions have traditionally been marketed in a variety of forms such as bar soaps, creams, lotions, and gels. Typically, these products must satisfy a number of criteria to be acceptable to consumers. These criteria include cleansing effectiveness, skin feel, mildness to skin, hair, and ocular mucosae, and lather volume. Ideal personal cleansers should gently cleanse the skin or hair, cause little or no irritation, and should not leave the skin or hair overly dry after frequent use.

Anionic surfactants are widely used in personal cleansing compositions. Many of these anionic surfactants contain elongated micelles and are viscoelastic, which is of foremost importance, especially in the design of shampoos and body washes. In most personal cleansing compositions, alkyl sulfate or alkyl ether sulfate as the anionic surfactants predominate.

The formulation of environmentally friendly personal cleansing compositions is becoming a major challenge for satisfying a new expectation of consumers, in particular that of ecologically designed and/or natural products. It becomes necessary to propose personal cleansing compositions substantially free of alkyl sulfate and alkyl ether sulfate, which have good cosmetic qualities, mainly in terms of viscosity, creamy lather, and clean skin feel.

Consumers prefer sulfate-substantially free personal cleansing compositions due to perceived mildness and desirable sensorial experience. There is an interest to provide personal cleansing products that comprise alternative mild surfactant systems with relatively improved ecotoxic or ecologically friendly environmental profile.

Personal cleansing compositions having a surfactant system comprising a fatty acyl isethionate surfactant and being substantially free of alkyl sulfate or alkyl ether sulfate type of surfactants have been developed. Fatty acyl isethionates are mild anionic surfactants highly desirable in personal cleansing products for hair or skin, because fatty acyl isethionates can lather well, are mild to the skin and have good emollient properties.

However, personal cleansing composition comprising fatty acyl isethionates may have phase stability challenges. Personal cleansing composition comprising fatty acyl isethionates may result in unstable personal cleansing compositions which can exhibit inconsistent rheology profiles. It has been observed phase separation of personal cleansing compositions comprising a fatty acyl isethionate and fatty acyl sarcosinate at 40° C. after 3 months. The phase separation might be attributed to the presence of fatty acid that has been generated from the hydrolysis of the fatty acyl isethionate. The initial wormlike and spherical micellar composition may separate into an upper lamellar phase and a lower micellar phase. Lamellar phases are typically relatively high ordered, surfactant-rich phases with relatively less water and low density such that the lamellar phases float.

Sulfate-substantially free personal cleansing compositions are also difficult to thicken sufficiently to afford the user good usage qualities. Two approaches are leveraged to attempt to thicken such formulas. One approach for instance is to use prominent levels of surfactants to benefit from the self-assembling properties of such ingredients. This approach is most common but it is also costly. The second approach for instance is to use elevated levels of rheology modifiers which can adversely impact the properties of the composition such as by decreasing the foam and case of distribution of the composition. A third approach consists in using a mixture of a fatty alkanolamide such as cocamide MEA and a hydrophobically modified ethoxylated methyl glucoside such as PEG-120 methyl glucose trioleate to reach the desired viscosity of the personal cleansing composition, see for instance PCT/CN2022/086854.

To overcome stability concern, pH can be increased which can significantly reduce the hydrolysis rate of fatty acyl isethionates. The surfactant system can be also optimized, see for instance PCT/CN2023/086792.

However, the rheology profile of the personal cleansing composition could still be optimized.

Hence, there is a need to provide a personal cleansing composition comprising an acyl isethionate surfactant, being substantially free of alkyl sulfate or alkyl ether sulfate type of surfactants and having a satisfactory consistent rheology profile without the use of any polymeric rheology modifiers or increased levels of electrolyte.

Also, there remains a need for a personal cleansing composition, which is effective at cleaning even while containing lower number of active surfactants than typical cleansing products, but also still possesses good esthetic properties such as good foam, and is thick and creamy in texture, is silky to the touch and affords conditioning.

### SUMMARY OF THE INVENTION

A personal cleansing composition is provided and comprises: a) a surfactant system, wherein the surfactant system comprises: i) from about 1.75% to about 15%, preferably from about 1.75% to about 10%, more preferably from about 1.75% to about 6%, most preferably from about 2.0% to about 3.0%, of an acyl isethionate surfactant by weight of the composition; wherein a chain length distribution in the acyl isethionate surfactant is such that:
C8 chain within the chain length distribution is between about 0.01 wt. % to about 3.5 wt. %, preferably between about 0.01 wt. % to about 3.0 wt. %, more preferably between about 0.01 wt. % to about 2.0 wt. %;
C10 chain within the chain length distribution is between about 0.01 wt. % to about 3.5 wt. %, preferably between about 0.01 wt. % to about 3.0 wt. %, more preferably between about 0.01 wt. % to about 2.0 wt. %;
C12 chain within the chain length distribution is between about 40 wt. % to about 80 wt. %, preferably between about 45 wt. % to about 77 wt. %, more preferably between about 51 wt. % to about 76 wt. %;
C14 chain within the chain length distribution is between about 10 wt. % to about 25 wt. %, preferably between about 15 wt. % to about 25 wt. %, more preferably between about 18 wt. % to about 22 wt. %;
C16 chain within the chain length distribution is between about 3 wt. % to about 14 wt. %, preferably between about 3 wt. % to about 14 wt. %, more preferably between about 3 wt. % to about 13 wt. %; and
C18 chain within the chain length distribution is between about 1 wt. % to about 14 wt. %, preferably between about 1 wt. % to about 10 wt. %, more preferably between about 1 wt. % to about 10 wt. %;
   b) wherein the pH is from about 5.5 to about 7, preferably from about 5.6 to about 6.5, more preferably from about 5.65 to about 6.0; and c) wherein the composition is substantially free of alkyl sulfate and alkyl ether sulfate type of surfactants.

A method of increasing the rheology of a personal cleansing composition is provided and comprises the step of forming a personal cleansing composition as disclosed herein.

### DETAILED DESCRIPTION OF THE INVENTION

### Definitions of Terms

In this document, the following definitions apply unless specifically stated otherwise.

All percentages are by weight (w/w) of the composition, unless otherwise specified. “% wt.” means percentage by weight. References to ‘parts’ e.g. a mixture of 1 part X and 3 parts Y, is a ratio by weight. All ratios or percentages are weight ratios or weight percentages unless specifically stated otherwise.

An “active composition” is the composition absent water, and an “active ingredient” is the ingredient absent its water.

“QS” or “QSP” means sufficient quantity for 100% or for 100 g. +/− indicates the standard deviation. All ranges are inclusive and combinable. The number of significant digits conveys neither a limitation on the indicated amounts nor on the accuracy of the measurements. All numerical amounts are understood to be modified by the word “about”.

All measurements are understood to be made at 25° C. and at ambient conditions, where “ambient conditions” means at 1 atmosphere (atm) of pressure and at 65% relative humidity, unless otherwise stated. “Relative humidity” refers to the ratio (stated as a percent) of the moisture content of air compared to the saturated moisture level at the same temperature and pressure. Relative humidity can be measured with a hygrometer, in particular with a probe hygrometer from VWR® International.

Herein “min” means “minute” or “minutes”. Herein “mol” means mole. Herein “g” following a number means “gram” or “grams”. “Ex.” means “example”. All amounts as they pertain to listed ingredients are based on the active level and do not include carriers or by-products that may be included in commercially available materials.

Herein, “comprising” means that other steps and other ingredients can be in addition. “Comprising” encompasses the terms “consisting of” and “consisting essentially of”. The compositions, methods, uses, and processes described herein can comprise, consist of, and consist essentially of the elements and limitations described herein, as well as any of the additional or optional ingredients, components, steps, or limitations described herein. Embodiments and aspects described herein may comprise or be combinable with elements, features or components of other embodiments and/or aspects despite not being expressly exemplified in combination, unless an incompatibility is stated.

As used herein, the articles including “a” and “an” when used in a claim, are understood to mean “one or more” of what is claimed or described.

The terms “include,” “includes,” and “including,” as used herein are meant to be non-limiting.

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the composition.

The term “free of” as used herein means that the composition comprises 0% of an ingredient by weight of the composition, thus no detectable amount of the stated ingredient.

The term “substantially free of” as used herein means less than 1.5%, less than 1.4%, less than 1.2%, less than 1%, less than 0.8%, less than 0.5%, less than 0.3%, or less than an immaterial amount of by weight of the composition, unless otherwise stated.

Herein “Comp. Ex.” or “C. Ex.” means comparative example; and “Ex.” means example.

The term “molecular weight” or “M.Wt.” as used herein refers to the weight average molecular weight unless otherwise stated. The weight average molecular weight can be measured by gel permeation chromatography (“GPC”).

The term “personal cleansing composition” as used herein refers to compositions intended for topical application to the hair and the skin, preferably to the skin, for cleansing.

The term “mixtures” as used herein is meant to include a simple combination of materials and any compounds that may result from their combination.

The term “room temperature” refers to a temperature of 25° C.

The term “rinse-off” as used herein means the intended product usage includes application to skin followed by rinsing and/or wiping the product from the skin within a few seconds to minutes of the application step. The product is generally applied and rinsed in the same usage event, for example, a shower or washing one's hands.

The term “derivative” as used herein refers to structures which are not shown but which one skilled in the art would understand are variations of the basic compound.

The methods as disclosed herein are cosmetic methods or non-therapeutic methods.

The personal cleansing products, methods and uses of the products, the structures and the respective compositions as described in the Summary or as described hereinbelow are for fulfilling the technical effects or goals as set out herein. These objects and other advantages as may be apparent to those skilled in the art can be achieved through personal cleansing products, methods and uses of the products, the structures and the respective compositions as described herein.

### Benefits

Personal cleansing compositions comprising a fatty acyl isethionate only; or with a fatty acyl sarcosinate may have shown phase stability challenges. Such phase separation is typically caused by the accumulation of fatty acid which is generated by the hydrolysis of the fatty acyl isethionate only or with fatty acyl sarcosinate overtime.

Phase separation has been observed for personal cleansing compositions comprising a fatty acyl isethionate at 40° C. after 3 months (see C. Ex. 1 for an example). The mechanism of the phase separation might lie in the hydrolysis of the fatty acyl isethionate and other anionic surfactants such as a fatty acyl sarcosinate catalyzed in an acidic medium (e.g. pH at 5).

Increasing the pH would slow down the hydrolysis kinetics (see C. Ex. 2 for an example), however, at a neutral pH, the zero-shear viscosity of the personal cleansing composition may collapse. Only a small portion of a deprotonated form of the anionic surfactant, e.g. fatty acyl isethionate or fatty acyl sarcosinate, is needed to form a stable wormlike micellar phase without the addition of any electrolyte. The pH of the personal cleansing composition cannot be raised far from the pKa of the anionic surfactant, otherwise, the viscosity peak is missed and the personal cleansing composition cannot meet the rheologic profile.

Recently, a new combination of ingredients was found to build the consistent viscosity of the personal cleansing composition (Sec C. Ex. 3 and C. Ex. 3 for examples).

Sodium cocoyl isethionate used in C. Ex. 1-4 is commercially available from Clariant as HOSTAPON SCI 85 C, for instance. In such material, the levels of each of C8 and C10 chains in this sodium cocoyl isethionate surfactant are above 3.5 wt. %.

However, there was still a need to further enhance the consistent viscosity of the personal cleansing composition. It has been found that the personal cleansing composition needs to include in a surfactant system at the recited pH, an acyl isethionate surfactant with a very specific chain length distribution. Each level of C8 and C10 chains in the acyl isethionate surfactant needs to be at the maximum about 3.5 wt. %. Also the over chain length distribution in the acyl isethionate surfactant needs to include some C14-C18 chains in specific proportions (See Ex. 1-4).

It has been provided that a personal cleansing composition needs to include a) a surfactant system, wherein the surfactant system comprises: i) from about 1.75% to about 15%, preferably from about 1.75% to about 10%, more preferably from about 1.75% to about 6%, most preferably from about 2.0% to about 3.0%, of an acyl isethionate surfactant by weight of the composition; wherein a chain length distribution in the acyl isethionate surfactant is such that: C8 chain within the chain length distribution is between about 0.01 wt. % to about 3.5 wt. %, preferably between about 0.01 wt. % to about 3.0 wt. %, more preferably between about 0.01 wt. % to about 2.0 wt. %; C10 chain within the chain length distribution is between about 0.01 wt. % to about 3.5 wt. %, preferably between about 0.01 wt. % to about 3.0 wt. %, more preferably between about 0.01 wt. % to about 2.0 wt. %; C12 chain within the chain length distribution is between about 40 wt. % to about 80 wt. %, preferably between about 45 wt. % to about 77 wt. %, more preferably between about 51 wt. % to about 76 wt. %; C14 chain within the chain length distribution is between about 10 wt. % to about 25 wt. %, preferably between about 15 wt. % to about 25 wt. %, more preferably between about 18 wt. % to about 22 wt. %; C16 chain within the chain length distribution is between about 3 wt. % to about 14 wt. %, preferably between about 3 wt. % to about 14 wt. %, more preferably between about 3 wt. % to about 13 wt. %; and C18 chain within the chain length distribution is between about 1 wt. % to about 14 wt. %, preferably between about 1 wt. % to about 10 wt. %, more preferably between about 1 wt. % to about 10 wt. %; b) wherein the pH is from about 5.5 to about 7, preferably from about 5.6 to about 6.5, more preferably from about 5.65 to about 6.0; and c) wherein the composition is substantially free of alkyl sulfate and alkyl ether sulfate type of surfactants.

Hence, the acyl isethionate surfactant has been now optimized to enable building the consistent viscosity of the personal cleansing composition. The personal cleansing composition is stable and shows a transparent homogeneous appearance overtime, through the shelf life.

The personal cleansing composition is thus thick and creamy in texture, is silky to the touch and affords conditioning.

### Surfactant System

A personal cleansing composition is provided and comprises a) a surfactant system, wherein the surfactant system comprises: i) from about 1.75% to about 15%, preferably from about 1.75% to about 10%, more preferably from about 1.75% to about 6%, most preferably from about 2.0% to about 3.0%, of an acyl isethionate surfactant by weight of the composition; wherein a chain length distribution in the acyl isethionate surfactant is such that: C8 chain within the chain length distribution is between about 0.01 wt. % to about 3.5 wt. %, preferably between about 0.01 wt. % to about 3.0 wt. %, more preferably between about 0.01 wt. % to about 2.0 wt. %; C10 chain within the chain length distribution is between about 0.01 wt. % to about 3.5 wt. %, preferably between about 0.01 wt. % to about 3.0 wt. %, more preferably between about 0.01 wt. % to about 2.0 wt. %; C12 chain within the chain length distribution is between about 40 wt. % to about 80 wt. %, preferably between about 45 wt. % to about 77 wt. %, more preferably between about 51 wt. % to about 76 wt. %; C14 chain within the chain length distribution is between about 10 wt. % to about 25 wt. %, preferably between about 15 wt. % to about 25 wt. %, more preferably between about 18 wt. % to about 22 wt. %; C16 chain within the chain length distribution is between about 3 wt. % to about 14 wt. %, preferably between about 3 wt. % to about 14 wt. %, more preferably between about 3 wt. % to about 13 wt. %; and C18 chain within the chain length distribution is between about 1 wt. % to about 14 wt. %, preferably between about 1 wt. % to about 10 wt. %, more preferably between about 1 wt. % to about 10 wt. %; b) wherein the pH is from about 5.5 to about 7, preferably from about 5.6 to about 6.5, more preferably from about 5.65 to about 6.0; and c) wherein the composition is substantially free of alkyl sulfate and alkyl ether sulfate type of surfactants.

The personal cleansing composition is substantially free of alkyl sulfate and/or alkyl ether sulfate type of surfactant. Namely, the personal cleansing composition comprises less than about 1.5%, or less than about 1.4%, or less than about 1.2%, or less than about 1%, or less than about 0.8%, or less than about 0.5%, or less than about 0.3%, or is free of alkyl sulfate and/or alkyl ether sulfate type of surfactant by weight of the composition.

Preferably, the personal cleansing composition comprises less than about 1.5%, or less than about 1.4%, or less than about 1.2%, or less than about 1%, or less than about 0.8%, or less than about 0.5%, or less than about 0.3%, or is free of any alkyl sulfate which comprises C12-C18 alkyl sulfate and/or any alkyl ether sulfate including alkyl glyceryl ether sulfates.

More preferably, the personal cleansing composition comprises less than about 1.5%, or less than about 1.4%, or less than about 1.2%, or less than about 1%, or less than about 0.8%, or less than about 0.5%, or less than about 0.3%, or is free of sodium lauryl sulfate.

Alternatively, the personal cleansing composition is free of alkyl sulfate and/or alkyl ether sulfate type of surfactant. Namely, the personal cleansing composition comprises 0% of alkyl sulfate and/or alkyl ether sulfate type of surfactant by weight of the composition, thus no detectable amount of alkyl sulfate and/or alkyl ether sulfate type of surfactant.

In that respect, the personal cleansing composition may not comprise any alkyl sulfate which comprises C12-C18 alkyl sulfate and/or any alkyl ether sulfate including alkyl glyceryl ether sulfates.

The personal cleansing composition may not comprise any alkyl ether sulfates which are those having the formula:

RO(CH₂CH₂O)ₙSO₃M

wherein R is an alkyl or alkenyl having 8 to 18 carbons, preferably 12 to 18 carbons, n has an average value of greater than at least 0.5, preferably between 2 and 3; and M is a solubilizing cation such as sodium, potassium, ammonium or substituted ammonium.

The personal cleansing composition may not comprise any ammonium and sodium lauryl ether sulfates.

If the personal cleansing composition does contain alkyl sulfate and/or alkyl ether sulfate type of surfactant, its content of such a weight proportion of: alkyl sulfates or alkyl ether sulfate type surfactant is less than or equal to the sum of about 0.6, more preferably less than or equal to the sum of about 0.2, even more preferably equal to 0.

### Acyl Isethionate Surfactant

The personal cleansing composition comprises a surfactant system. The surfactant system comprises from about 1.75% to about 15%, preferably from about 1.75% to about 10%, more preferably from about 1.75% to about 6%, most preferably from about 2.0% to about 3.0% of an acyl isethionate surfactant by weight of the composition.

Alternatively, the personal cleansing composition may comprise a surfactant system, wherein the surfactant system comprises from about 1.75% to about 3.0%, preferably from about 2.0% to about 3.0%, more preferably from about 2.25% to about 3.0%, most preferably from about 2.5% to about 3.0% of an acyl isethionate surfactant by weight of the composition.

The concentrations mentioned here are total concentration ranges in case more than one acyl isethionate surfactant is present. The specified ranges are provided by weight and relate to the total weight of the personal cleansing composition. The concentrations mentioned hereinbefore apply to any chain length distribution in the acyl isethionate surfactant defined herein.

The acyl isethionate surfactant has a specific chain length distribution. The acyl isethionate may be defined as an isethionate according to the general Formula (10):

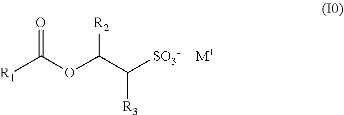

wherein R₁ is a saturated or unsaturated, straight or branched, alkyl or alkenyl chain with from 8 to 18 carbon atoms, R₂ and R₃ are each independently H or (C₁-C₄) alkyl, preferably R₂ and R₃ are H; and M⁺ is an alkali metal, preferably lithium, sodium, potassium; or M⁺ is an alkali-earth metal, preferably magnesium; or M⁺ is an ammonium or a substituted ammonium cation. An Acyl group is thus “R₁—C(O)—” moiety wherein R₁ includes C8-18 chains.

A chain length distribution in the acyl isethionate surfactant is such that:
C8 chain within the chain length distribution is between about 0.01 wt. % to about 3.5 wt. %, preferably between about 0.01 wt. % to about 3.0 wt. %, more preferably between about 0.01 wt. % to about 2.0 wt. %;
C10 chain within the chain length distribution is between about 0.01 wt. % to about 3.5 wt. %, preferably between about 0.01 wt. % to about 3.0 wt. %, more preferably between about 0.01 wt. % to about 2.0 wt. %;
C12 chain within the chain length distribution is between about 40 wt. % to about 80 wt. %, preferably between about 45 wt. % to about 77 wt. %, more preferably between about 51 wt. % to about 76 wt. %;
C14 chain within the chain length distribution is between about 10 wt. % to about 25 wt. %, preferably between about 15 wt. % to about 25 wt. %, more preferably between about 18 wt. % to about 22 wt. %;
C16 chain within the chain length distribution is between about 3 wt. % to about 14 wt. %, preferably between about 3 wt. % to about 14 wt. %, more preferably between about 3 wt. % to about 13 wt. %; and
C18 chain within the chain length distribution is between about 1 wt. % to about 14 wt. %, preferably between about 1 wt. % to about 10 wt. %, more preferably between about 1 wt. % to about 10 wt. %.

The concentrations for each chain mentioned here are by weight of the acyl isethionate surfactant and can be measured according to the Fatty acid chain length distribution Test Method as disclosed herein.

In other words, the chain length distribution in the acyl isethionate surfactant is such that: C8 chain within the chain length distribution is between about 0.01 wt. % to about 3.5 wt. %; C10 chain within the chain length distribution is between about 0.01 wt. % to about 3.5 wt. %; C12 chain within the chain length distribution is between about 40 wt. % to about 80 wt. %; C14 chain within the chain length distribution is between about 10 wt. % to about 25 wt. %; C16 chain within the chain length distribution is between about 3 wt. % to about 14 wt. %; and C18 chain within the chain length distribution is between about 1 wt. % to about 14 wt. %.

Higher level of C8 and C10 chains above 3.5 wt. % can lead to insufficient viscosity building.

Preferably, the chain length distribution in the acyl isethionate surfactant may be such that: C8 chain within the chain length distribution is between about 0.01 wt. % to about 3.0 wt. %; C10 chain within the chain length distribution is between about 0.01 wt. % to about 3.0 wt. %; C12 chain within the chain length distribution is between about 45 wt. % to about 77 wt. %; C14 chain within the chain length distribution is between about 15 wt. % to about 25 wt. %; C16 chain within the chain length distribution is between about 3 wt. % to about 14 wt. %; and C18 chain within the chain length distribution is between about 1 wt. % to about 10 wt. %.

More preferably, the chain length distribution in the acyl isethionate surfactant may be such that: C8 chain within the chain length distribution is between about 0.01 wt. % to about 2.0 wt. %; C10 chain within the chain length distribution is between about 0.01 wt. % to about 2.0 wt. %; C12 chain within the chain length distribution is between about 51 wt. % to about 76 wt. %; C14 chain within the chain length distribution is between about 18 wt. % to about 22 wt. %; C16 chain within the chain length distribution is between about 3 wt. % to about 13 wt. %; and C18 chain within the chain length distribution is between about 1 wt. % to about 10 wt. %.

Alternatively, the chain length distribution in the acyl isethionate surfactant is such that: C8 chain within the chain length distribution is between about 1.9 wt. % to about 3.5 wt. %; C10 chain within the chain length distribution is between about 1.9 wt. % to about 3.5 wt. %; C12 chain within the chain length distribution is between about 40 wt. % to about 80 wt. %; C14 chain within the chain length distribution is between about 10 wt. % to about 25 wt. %; C16 chain within the chain length distribution is between about 3 wt. % to about 13 wt. %; and C18 chain within the chain length distribution is between about 1 wt. % to about 14 wt. %.

Preferably, the chain length distribution in the acyl isethionate surfactant may be such that: C8 chain within the chain length distribution is between about 1.9 wt. % to about 3.5 wt. %; C10 chain within the chain length distribution is between about 1.9 wt. % to about 3.5 wt. %; C12 chain within the chain length distribution is between about 45 wt. % to about 77 wt. %; C14 chain within the chain length distribution is between about 15 wt. % to about 25 wt. %; C16 chain within the chain length distribution is between about 3 wt. % to about 14 wt. %; and C18 chain within the chain length distribution is between about 1 wt. % to about 10 wt. %.

More preferably, the chain length distribution in the acyl isethionate surfactant may be such that: C8 chain within the chain length distribution is between about 2.0 wt. % to about 3.5 wt. %; C10 chain within the chain length distribution is between about 2.0 wt. % to about 3.5 wt. %; C12 chain within the chain length distribution is between about 45 wt. % to about 76 wt. %; C14 chain within the chain length distribution is between about 16 wt. % to about 24 wt. %; C16 chain within the chain length distribution is between about 3 wt. % to about 13 wt. %; and C18 chain within the chain length distribution is between about 1 wt. % to about 10 wt. %.

In any cases, the chain length distribution in the acyl isethionate surfactant may be substantially free of C18:1 and C18:2 chains. Namely, the chain length distribution in the acyl isethionate surfactant may comprise less than about 0.1 wt. % or less than 0.05 wt. % or less than 0.02 wt. % or less than 0.01 wt. % or free of C18:1 and C18:2 chains. C18:1 is a C18 chain with one double bond. C18:2 is a C18 chain with two double bonds.

The acyl isethionate surfactant may be a sodium, potassium or ammonium acyl isethionate.

The acyl isethionate surfactant may be selected from the group consisting of sodium cocoyl isethionate, sodium cocoyl methyl isethionate, a blend of stearic acid and sodium cocoyl isethionate, ammonium cocoyl isethionate, ammonium cocoyl methyl isethionate, and mixtures thereof.

The acyl isethionate surfactant may be preferably selected from the group consisting of sodium cocoyl isethionate, ammonium cocoyl isethionate, and mixtures thereof.

The acyl isethionate surfactant may more preferably comprise sodium cocoyl isethionate.

In that aspect, the personal cleansing composition may comprise a surfactant system, wherein the surfactant system comprises from about 1.75% to about 15%, preferably from about 1.75% to about 10%, more preferably from about 1.75% to about 6%, most preferably from about 2.0% to about 3.0% of sodium cocoyl isethionate by weight of the composition.

Alternatively, the personal cleansing composition may comprise a surfactant system, wherein the surfactant system comprises from about 1.75% to about 3.0%, preferably from about 2.0% to about 3.0%, more preferably from about 2.25% to about 3.0%, most preferably from about 2.5% to about 3.0% of sodium cocoyl isethionate surfactant by weight of the composition.

Alternatively, the personal cleansing composition may comprise a surfactant system, wherein the surfactant system comprises: from 1.75% to 2.75%, preferably from 2.0% to 2.5%, more preferably from 2.25% to 2.5%, most preferably from 2.35% to 2.5%, of sodium cocoyl isethionate by weight of the composition.

Fatty acyl isethionates surfactants are typically prepared by the reaction of an isethionate salt such as metal or ammonium isethionate and an a saturated or unsaturated, straight or branched, alkyl or alkenyl chain fatty acid having from 6 to 30 carbon atoms, preferably from 8 to 22 carbon atoms, more preferably from 6 to 18 carbon atoms. Depending on the processing conditions used, the resulting fatty acyl isethionate surfactant can be a mixture of 45 to 95% by weight of fatty acyl isethionates and 0 to 40 wt. % of free fatty acids, in addition to isethionates salts, typically less than 5 wt. %, and trace (less than 2 wt. %) of other impurities, by total weight of the resulting fatty acyl isethionate surfactant. A mixture of aliphatic fatty acids may be used for the preparation of commercial fatty acyl isethionates surfactants.

Preparation of acyl isethionate surfactants with different chain length distributions is well-known, see for instance U.S. Pat. No. 5,384,421 A; 5,681,980 A; or 6,069,262 A, each of which is incorporated herein by reference.

The acyl isethionate surfactant may comprise from about 85 wt. % to about 99.95 wt. % of an acyl isethionate, preferably sodium cocoyl isethionate, and from about 0.05 wt. % to about 4 wt. % of a free fatty acid. Preferably, the acyl isethionate surfactant may comprise from about 85 wt. % to about 97.95 wt. % of an acyl isethionate, preferably sodium cocoyl isethionate, and from about 2 wt. % to about 4 wt. % of a free fatty acid.

The free fatty acid useful herein are those having from about 8 to about 18 carbon atoms. These fatty acids are saturated or unsaturated and can be straight or branched chain acids. Suitable fatty acids include, for example, caprylic acid, capric acid, lauric acid, myristic acid, palmitic acid, stearic acid, oleic acid, linoleic acid and mixtures thereof.

The surfactant system may comprise an additional isethionate surfactant, wherein the additional isethionate surfactant is selected from the group consisting of sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, and mixtures thereof.

### Fatty Acyl Sarcosinate

The surfactant system may comprise from about 1.75% to about 10%, preferably from about 2.0% to about 5.0%, more preferably from about 2.0% to about 4.0%, of a fatty acyl sarcosinate surfactant by weight of the composition.

Alternatively, the surfactant system may comprise from about 1.75% to about 3.0%, preferably from about 2.0% to about 2.5%, more preferably from about 2.25% to about 2.5%, most preferably from about 2.35% to about 2.5%, of a fatty acyl sarcosinate surfactant by weight of the composition.

The fatty acyl sarcosinate surfactant may be a sarcosinate according to the general Formula (I):

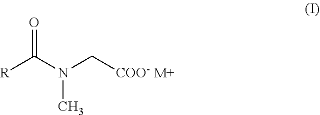

wherein R is a saturated or unsaturated, straight or branched or alkenyl, preferably alkyl chain with 7 to 17 carbon atoms, preferably with 9 to 13 carbon atoms and M⁺ is H, a sodium, potassium or ammonium cation.

The fatty acyl sarcosinate surfactant may be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroyl glutamate/lauroyl sarcosinate, lauroyl sarcosinate, isopropyl lauroyl sarcosinate, potassium cocoyl sarcosinate, potassium lauroyl sarcosinate, sodium oleoyl sarcosinate, sodium palmitoyl sarcosinate, TEA-lauroyl sarcosinate, TEA-oleoyl sarcosinate, TEA-palm kernel sarcosinate, and mixtures thereof. For instance, TEA-cocoyl sarcosinate is the triethanolamine salt of cocoyl sarcosine.

Preferably, the fatty acyl sarcosinate surfactant may be selected from the group consisting of sodium lauroyl sarcosinate, sodium myristoyl sarcosinate, sodium cocoyl sarcosinate, and mixtures thereof.

The fatty acyl sarcosinate surfactant may most preferably comprise sodium lauroyl sarcosinate.

In that aspect, the personal cleansing composition may comprise a surfactant system, wherein the surfactant system comprises from about 1.75% to about 10%, preferably from about 2.0% to about 5.0%, more preferably from about 2.0% to about 4.0%, of sodium lauroyl sarcosinate surfactant by weight of the composition.

Alternatively, the personal cleansing composition may comprise a surfactant system, wherein the surfactant system comprises from about 1.75% to about 3.0%, preferably from about 2.0% to about 2.5%, more preferably from about 2.25% to about 2.5%, most preferably from about 2.35% to about 2.5%, of sodium lauroyl sarcosinate by weight of the composition.

By decreasing slightly the level of the fatty acyl sarcosinate in the personal cleansing composition, a consistent rheology profile could be obtained.

The weight ratio of fatty acyl sarcosinate surfactant to acyl isethionate surfactant may be above about 0.5:1, or from about 1.75:1 to about 1:0.65, preferably from about 1.33:1 to about 1:0.75, more preferably from about 1.2:1 to about 1:0.95.

The composition may comprise sodium lauroyl sarcosinate and sodium cocoyl isethionate at a weight ratio sarcosinate to isethionate above about 0.5:1, or from about 1.75:1 to about 1:0.65, preferably from about 1.33:1 to about 1:0.75, more preferably from about 1.2:1 to about 1:0.95.

In that aspect, by selecting the respective weight ratio, it is possible to enable building and improving the rheology profile of the personal cleansing composition even further.

### Additional Anionic Surfactant

The composition may comprise one or more additional anionic surfactant not being an acyl isethionate or a fatty acyl sarcosinate.

The surfactant system may comprise from about 0.5% to about 25%, preferably from about 1% to about 20%, more preferably from about 5% to about 15% of the one or more additional anionic surfactant not being a fatty acyl isethionate or a fatty acyl sarcosinate by weight of the composition.

The one or more additional anionic surfactant not being an acyl isethionate or a fatty acyl sarcosinate may be selected from the group consisting of sulfosuccinates, sulfonates, sulfoacetates, acyl glycinates, acyl alaninates, acyl glutamates, lactates, lactylates, taurates, and mixtures thereof.

Non-limiting examples of sulfosuccinate surfactants can include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid, dioctyl esters of sodium sulfosuccinic acid, and combinations thereof.

Non-limiting examples of sulfonates can include alpha olefin sulfonates, linear alkylbenzene sulfonates, sodium laurylglucosides hydroxypropylsulfonate, and combinations thereof.

Non-limiting examples of sulfoacetates can include sodium lauryl sulfoacetate, ammonium lauryl sulfoacetate, and combination thereof.

Non-limiting examples of acyl glycinates can include sodium cocoyl glycinate, sodium lauroyl glycinate, and combination thereof.

Non-limiting example of acyl alaninates can include sodium cocoyl alaninate, sodium lauroyl alaninate, sodium N-dodecanoyl-1-alaninate, and combinations thereof.

Non-limiting examples of acyl glutamates can be selected from the group consisting of sodium cocoyl glutamate, disodium cocoyl glutamate, ammonium cocoyl glutamate, diammonium cocoyl glutamate, sodium lauroyl glutamate, disodium lauroyl glutamate, sodium cocoyl hydrolyzed wheat protein glutamate, disodium cocoyl hydrolyzed wheat protein glutamate, potassium cocoyl glutamate, dipotassium cocoyl glutamate, potassium lauroyl glutamate, dipotassium lauroyl glutamate, potassium cocoyl hydrolyzed wheat protein glutamate, dipotassium cocoyl hydrolyzed wheat protein glutamate, sodium capryloyl glutamate, disodium capryloyl glutamate, potassium capryloyl glutamate, dipotassium capryloyl glutamate, sodium undecylenoyl glutamate, disodium undecylenoyl glutamate, potassium undecylenoyl glutamate, dipotassium undecylenoyl glutamate, disodium hydrogenated tallow glutamate, sodium stearoyl glutamate, disodium stearoyl glutamate, potassium stearoyl glutamate, dipotassium stearoyl glutamate, sodium myristoyl glutamate, disodium myristoyl glutamate, potassium myristoyl glutamate, dipotassium myristoyl glutamate, sodium cocoyl/hydrogenated tallow glutamate, sodium cocoyl/palmoyl/sunfloweroyl glutamate, sodium hydrogenated tallowoyl glutamate, sodium olivoyl glutamate, disodium olivoyl glutamate, sodium palmoyl glutamate, disodium palmoyl glutamate, TEA-cocoyl glutamate, TEA-hydrogenated tallowoyl glutamate, TEA-lauroyl glutamate, and mixtures thereof.

Non-limiting example of lactates can include sodium lactate.

Non-limiting examples of lactylates can include sodium lauroyl lactylate, sodium cocoyl lactylate, and combination thereof.

Non-limiting examples of acyl taurates can include sodium methyl cocoyl taurate, sodium methyl lauroyl taurate, sodium methyl oleoyl taurate, and combinations thereof.

In that case, alkyl is defined as a saturated or unsaturated, straight or branched alkyl chain with 6 to 30 carbon atoms, preferably with 8 to 22 carbon atoms, more preferably with 9 to 18 carbon atoms. In that case, acyl is defined as of formula R—C(O)—, wherein R is a saturated or unsaturated, straight or branched alkyl or alkenyl, preferably alkyl chain with 6 to 30 carbon atoms, preferably with 8 to 22 carbon atoms, more preferably with 9 to 18 carbon atoms.

### Zwitterionic Surfactant

The surfactant system may comprise from about 3% to about 20%, preferably from about 5% to about 15%, more preferably from about 5% to about 10%, of a zwitterionic surfactant by weight of composition, wherein the zwitterionic surfactant comprises a betaine.

Alternatively, the surfactant system may comprise from about 7.75% to about 10.5%, preferably from about 8.0% to about 10.5%, more preferably from about 8.75% to about 10.5%, most preferably from about 9.0% to about 10% of a zwitterionic surfactant by weight of composition, wherein the zwitterionic surfactant comprises a betaine.

The zwitterionic surfactant may comprise an alkyl betaine or an alkyl amidopropyl betaine; or a sulfobetaine.

Examples of betaine zwitterionic surfactants may include coco dimethyl carboxymethyl betaine, cocoamidopropyl betaine (CAPB), coco-betaine, lauramidopropyl betaine (LAPB), oleyl betaine, lauryl dimethyl carboxymethyl betaine, lauryl dimethyl alpha-carboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, and mixtures thereof.

Examples of sulfobetaines may include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

More preferably, the betaine may be selected from the group consisting of cocamidopropyl betaine, lauramidopropyl betaine, coco betaine, and mixtures thereof.

The surfactant system may comprise from about 3% to about 20%, preferably from about 5% to about 15%, more preferably from about 5% to about 10%, of cocamidopropyl betaine by weight of the composition.

Alternatively, the surfactant system may comprise from 7.75% to 10.5%, preferably from 8.0% to 10.5%, more preferably from 8.75% to 10.5%, most preferably from 9.0% to 10% of cocamidopropyl betaine by weight of the composition.

In that aspect, the phase stability of the personal cleansing composition can be further enhanced.

The weight ratio of betaine to fatty acyl sarcosinate surfactant may be equal or above about 2:1, or from about 3.4:1 to about 3.8:1, preferably from about 3.45:1 to about 3.7:1, more preferably from about 3.5:1 to about 3.65:1.

The composition may comprise cocamidopropyl betaine and sodium lauroyl sarcosinate at a weight ratio equal or above about 2:1, or from about 3.4:1 to about 3.8:1, preferably from about 3.45:1 to about 3.7:1, more preferably from about 3.5:1 to about 3.65:1.

The weight ratio of betaine to fatty acyl sarcosinate surfactant can help to further enhance the rheology profile of the personal cleansing composition systematically. The weight ratio of betaine to fatty acyl sarcosinate surfactant has been optimized to enable building the viscosity of the personal cleansing composition at the recited pH.

In an aspect, the surfactant system may comprise: from about 1.75% to about 15%, preferably from about 1.75% to about 10%, more preferably from about 1.75% to about 6%, most preferably from about 2.0% to about 3.0%, of the acyl isethionate surfactant by weight of the composition; from about 1.75% to about 10%, preferably from about 2.0% to about 5.0%, more preferably from about 2.0% to about 4.0%, of a fatty acyl sarcosinate surfactant by weight of the composition; from about 3% to about 20%, preferably from about 5% to about 15%, more preferably from about 5% to about 10%, of a zwitterionic surfactant by weight of composition, wherein the zwitterionic surfactant comprises a betaine.

Preferably, the surfactant system may comprise: from about 1.75% to about 15%, preferably from about 1.75% to about 10%, more preferably from about 1.75% to about 6%, most preferably from about 2.0% to about 3.0%, of sodium cocoyl isethionate by weight of the composition; from about 1.75% to about 10%, preferably from about 2.0% to about 5.0%, more preferably from about 2.0% to about 4.0%, of sodium lauroyl sarcosinate by weight of the composition; from about 3% to about 20%, preferably from about 5% to about 15%, more preferably from about 5% to about 10%, of cocamidopropyl betaine or lauramidopropyl betaine or coco betaine.

In another aspect, the surfactant system may comprise: from about 2.0% to about 3.0%, preferably from about 2.5% to about 3.0% of the acyl isethionate surfactant by weight of the composition; from about 1.75% to about 4.0%, preferably from about 2.5% to about 4.0%, of the fatty acyl sarcosinate surfactant by weight of the composition; from about 7.2% to about 10%, preferably from about 8.0% to about 10% of the betaine.

Preferably, the surfactant system may comprise: from about 2.0% to about 3.0%, preferably from about 2.5% to about 3.0% of sodium cocoyl isethionate by weight of the composition; from about 1.75% to about 4.0%, preferably from about 2.5% to about 4.0%, of sodium lauroyl sarcosinate by weight of the composition; from about 7.2% to about 10%, preferably from about 8.0% to about 10% of cocamidopropyl betaine.

Alternatively, the personal cleansing composition may comprise a surfactant system, wherein the surfactant system comprises from about 1.75% to about 3.0%, preferably from about 2.0% to about 3.0%, more preferably from about 2.25% to about 3.0%, most preferably from about 2.5% to about 3.0% of an acyl isethionate surfactant by weight of the composition; from about 1.75% to about 3.0%, preferably from about 2.0% to about 2.5%, more preferably from about 2.25% to about 2.5%, most preferably from about 2.35% to about 2.5%, of a fatty acyl sarcosinate surfactant by weight of the composition; from about 7.75% to about 10.5%, preferably from about 8.0% to about 10.5%, more preferably from about 8.75% to about 10.5%, most preferably from about 9.0% to about 10% of a zwitterionic surfactant by weight of composition, wherein the zwitterionic surfactant comprises a betaine.

Preferably, the personal cleansing composition may comprise a surfactant system, wherein the surfactant system comprises from about 1.75% to about 3.0%, preferably from about 2.0% to about 3.0%, more preferably from about 2.25% to about 3.0%, most preferably from about 2.5% to about 3.0% of sodium cocoyl isethionate by weight of the composition; from about 1.75% to about 3.0%, preferably from about 2.0% to about 2.5%, more preferably from about 2.25% to about 2.5%, most preferably from about 2.35% to about 2.5%, of sodium lauroyl sarcosinate by weight of the composition; from about 7.75% to about 10.5%, preferably from about 8.0% to about 10.5%, more preferably from about 8.75% to about 10.5%, most preferably from about 9.0% to about 10% of cocamidopropyl betaine.

### Additional Co-Surfactant

The surfactant system may include an additional co-surfactant, wherein the additional co-surfactant comprises an amphoteric surfactant. Suitable amphoteric surfactants can include those described in U.S. Pat. Nos. 5,104,646 and 5,106,609, each of which is incorporated herein by reference.

Amphoteric surfactants can include those that can be broadly described as derivatives of aliphatic secondary and tertiary amines in which an aliphatic radical can be straight or branched chain and wherein an aliphatic substituent can contain from 8 to 18 carbon atoms such that one carbon atom can contain an anionic water solubilizing group, e.g., carboxy, sulfonate, phosphate, or phosphonate. Examples of compounds falling within this definition can be sodium 3-dodecyl-aminopropionate, sodium 3-dodecylaminopropane sulfonate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378, each of which is incorporated herein by reference.

The amphoteric surfactant included in the personal cleansing composition described herein may be preferably selected from the group consisting of sodium lauroamphoacetate, sodium cocoamphoacetate, disodium lauroamphoacetate, disodium cocodiamphoacetate, and mixtures thereof.

### pH

The pH of the personal cleansing composition is from 5.5 to 7, preferably from 5.5 to 6.5, more preferably from 5.65 to 6.0. Increasing the pH can help for preventing hydrolysis of the fatty acyl isethionate and the fatty acyl sarcosinate. pH may be measured according to the Product pH Measurement Test Method, described hereafter.

A variety of compounds may be used to adjust the pH value of a composition. Such suitable compounds can include, but are not limited to, citric acid, acetic acid, hydrochloric acid, sodium hydroxide, magnesium hydroxide, triethylamine, diethylamine, ethylamine, monoethanol amine, and any mixtures thereof. The personal cleansing composition may comprise greater than about 0% to about 2% of the pH adjusting agent by weight of the composition, preferably wherein the pH adjusting agent comprises citric acid.

By rising up the pH of the personal cleansing composition, the kinetics of generating fatty acids from the hydrolysis of the fatty acyl isethionate or the fatty acyl sarcosinate is slowed down. Increasing the pH of the personal cleansing composition can help to prevent phase separation of the personal cleansing composition. Then, the surfactant levels and/or can be optimized as described herein for building and improving the rheology profile of the personal cleansing composition.

### Electrolyte

The personal cleansing composition may further comprise from about 0.05 to about 5%, preferably from about 1% to about 5%, more preferably from about 2% to about 5%, even more preferably from about 2% to about 4% of an electrolyte by weight of the composition. The addition of an electrolyte can help to elongate the micelles of the surfactant system and to improve further the viscosity of the composition if needed.

The electrolyte may be selected from the group of sodium or potassium citrate, calcium chloride, calcium bromide, zinc chloride, barium chloride, calcium nitrate, potassium chloride, sodium chloride, potassium iodide, sodium bromide, ammonium bromide, sodium sulfate, and mixtures thereof.

The electrolyte may be preferably selected from the group of sodium or potassium citrate, calcium chloride, potassium chloride, sodium chloride, and mixtures thereof.

Most preferably, the personal cleansing composition may further comprise from about 2% to about 4% of an electrolyte by weight of the composition, wherein the electrolyte is sodium chloride.

The electrolyte itself can increase the initial viscosity, however, the inventors have found that the rheology profile of the personal cleansing composition could be optimized at a constant level of an electrolyte (2 wt. % sodium chloride in Ex. 1-4) or even without any addition of an electrolyte by adjusting the chain length distribution of the acyl isethionate surfactant, the surfactant levels and/or the specific surfactant weight ratios.

The personal cleansing composition may not comprise any rheology polymeric modifiers Examples of rheology polymeric modifiers may be but not limited to: sodium polyacrylate, acrylates copolymer, Acrylates/Vinyl Isodecanoate Crosspolymer, Acrylates/C10-30 Alkyl Acrylate Crosspolymer, Acrylates/C10-30 alkyl acrylate crosspolymer comprising stearyl side chains with less than about 1% Hydrophobic modification, acrylates/C10-30 alkyl acrylate crosspolymer including octyl side chains with less than about 5% Hydrophobic modification, Ammonium Acryloyldimethyltaurate/Beheneth-25 Methacrylate Crosspolymer, Acrylates/Beheneth-25 Methacrylate Copolymer, Acrylates/Steareth-20 Methacrylate Copolymer, and Acrylates/Steareth-20 Methacrylate Crosspolymer, PEG-150/Decyl Alcohol/SMDI Copolymer, PEG-150/stearyl alcohol/SMDI copolymer, hydroxypropyl starch phosphate, distarch phosphate, sodium carboxymethyl starch, hydroxypropyl starch phosphate, starch, Tapioca starch, xanthan gum, gellan gum, carboxymethyl cellulose, carboxymethyl hydroxyethyl cellulose, hydroxypropyl methyl cellulose, guar gum, hydroxypropyl guar, sodium alginate, and mixtures thereof.

The personal cleansing composition may be a body wash and may not comprise any cationic deposition polymers. Nonlimiting examples of cationic deposition polymers for use in the body wash include cationic cellulose derivatives. Cationic cellulose polymers are the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers, e.g. KG-30M. Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Aqualon.

The personal cleansing composition may not comprise a direct dye and/or any oxidative dye precursors.

### Fragrance Component

The personal cleansing composition may further comprise from about 0.01% to about 2% of a fragrance component by weight of the composition, preferably from about 0.1% to about 1.75% of a fragrance component by weight of the composition, more preferably from about 0.5% to about 1.6% of a fragrance component by weight of the composition, even more preferably from about 0.8% to about 1.0% of a fragrance component by weight of the composition.

Typically the fragrance component may be a blend of perfumes and aroma chemicals. As used herein, “fragrance” is used to indicate any odoriferous material.

A wide variety of chemicals are known as fragrances, including alcohols, aldehydes, ketones, and esters. Non-limiting examples of the fragrances useful herein include pro-fragrances such as acetal pro-fragrances, ketal pro-fragrances, ester pro-fragrances, hydrolyzable inorganic-organic pro-fragrances, and mixtures thereof. The fragrances may be released from the pro-fragrances in a number of ways. For example, the fragrance may be released as a result of simple hydrolysis, or by a shift in an equilibrium reaction, or by a pH-change, or by enzymatic release. The fragrances herein may be relatively simple in their chemical make-up, comprising a single chemical, or may comprise highly sophisticated complex mixtures of natural and synthetic chemical components, all chosen to provide any desired odor.

The fragrances may have a boiling point (BP) of 500° C. or lower, 400° C. or lower, or 350° C. or lower. The BP of many fragrances are disclosed in Perfume and Flavor Chemicals (Aroma Chemicals), Steffen Arctander (1969). The ClogP value of the fragrances may be 0.1 or greater, 0.5 or greater, 1.0 or greater, and 1.2 or greater. As used herein, “ClogP” means the logarithm to the base 10 of the octanol/water partition coefficient. The ClogP may be readily calculated from a program called “CLOGP” which is available from Daylight Chemical Information Systems Inc., Irvine Calif., USA. Octanol/water partition coefficients are described in more detail in U.S. Pat. No. 5,578,563, each of which is incorporated herein by reference.

Suitable fragrances are also disclosed in U.S. Pat. Nos. 4,145,184, 4,209,417, 4,515,705, and 4,152,272, each of which is incorporated herein by reference. Non.-limiting examples of fragrances include animal fragrances such as musk oil, civet, castoreurn, ambergris, plant fragrances such as nutmeg extract, cardomon extract, ginger extract, cinnamon extract, patchouli oil, geranium oil, orange oil, mandarin oil, orange Hower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, and mixtures thereof.

Other examples of suitable fragrances include, but are not limited to, chemical substances such as acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, borneol, butyl acetate, camphor, carbitol, cinnamaldehyde, cinnamyl acetate, cinnamyl alcohol, cis-3-hexanol and ester derivatives, cis-3-bexenyl methyl carbonate, citral, citronnellol and ester derivatives, cumin aldehyde, cyclamen aldehyde, cyclogalbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltoiide, fenchone, fruity esters such as ethyl 2-methyl butyrate, galaxolide, geraniol and ester derivatives, helional, 2-heptonone, hexenol, α-hexylcinnamic aldehyde, hydroxycitronellal, indole, isoamyl acetate, isoeugenol acetate, ionones, isocugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dibydrojasmonate, methyl cugenol, methyl ionone, methyl-α-naphthyl ketone, methylphenylcarbinyl acetate, mugetanol, γ-nonalactone, octanal, phenyl ethyl acetate, phenylacetaldehyde dimethyl acetate, phenoxyethyl isobutyrate, phenyl ethyl alcohol, pinenes, sandalore, santaiol, stemone, thymol, terpenes, triplal, triethyl citrate, 3,3,5-trimethylcyclohexanol, γ-undecalactone, undecenal, vanillin, veloutone, verdox, and mixtures thereof.

In the personal cleansing composition, the fragrance component may comprise ketone and/or aldehyde fragrance components. In addition, the fragrance component may further comprise any fragrances as set out just above.

Ketone fragrance components may be selected from alicyclic ketones such a β-ionone, terpene ketones such as l-carvone, and macrocyclic ketones such as cyclopentadecanone.

Aldehyde fragrance components may be selected from fatty aldehydes such as 2,6-nonadienal, terpene aldehydes such as citral, and aromatic aldehydes such as α-hexylcinnamic aldehyde, cinnamaldehyde.

Thus, preferably the fragrance component may comprise ketone and/or aldehyde fragrance components, wherein the ketone and/or aldehyde fragrance components may be selected from the group consisting of acetophenone, adoxal, aldehyde C-12, aldehyde C-14, aldehyde C-18, α-amylcinnamic aldehyde, anisaldehyde, benzaldehyde, camphor, cinnamaldehyde, citral, cumin aldehyde, cyclamen aldehyde, damascones, fenchone, helional, 2-heptonone, α-hexylcinnamic aldehyde, hydroxycitronellal, ionones, lilial, lyral, melonal, p-methylacetophenone, methyl cedrylone, methyl ionone, methyl-α-naphthyl ketone, γ-nonalactone, octanal, phenylacetaldehyde dimethyl acetate, triplal, γ-undecalactone, undecenal, vanillin, veloutone, and mixtures thereof.

### Preservative

The personal cleansing composition may comprise from about 0.01% to about 1.0%, preferably from about 0.02% to about 0.4%, more preferably from about 0.05% to about 0.2%, most preferably from about 0.05% to about 0.1% of a preservative by weight of the composition.

The preservative may include a salicylate salt and a benzoate salt, wherein a total amount of the salicylate salt and the benzoate salt is from about 0.2% to about 0.9%, preferably from about 0.5% to about 0.85%, more preferably from about 0.75% to about 0.85%, by weight of the composition.

The weight ratio of the salicylate salt to the benzoate salt may be from about 1:1.10 to about 1:1.20, preferably from about 1:1.125 to about 1:1.175.

The salicylate salt may be sodium salicylate. The benzoate salt may be sodium benzoate.

### Optional Ingredients

As can be appreciated, the compositions described herein may include a variety of optional components to tailor the properties and characteristics of the composition. As can be appreciated, suitable optional components are well known and can generally include any components which are physically and chemically compatible with the essential components of the compositions described herein. Optional components should not otherwise unduly impair product stability, aesthetics, or performance. Individual concentrations of optional components can generally range from 0.001% to 10%, by weight of the composition. Optional components can be further limited to components which will not impair the clarity of a translucent composition.

Still, the personal cleansing composition may not include or may be free of direct dyes, oxidative dyes, parabens, or mixtures thereof.

Optional components may include, but are not limited to, conditioning agents (including hydrocarbon oils, fatty esters, silicones), anti-dandruff actives, and chelating agents. Additional suitable optional ingredients include but are not limited to particles, anti-microbials, foam boosters, anti-static agents, moisturizing agents, propellants, self-foaming agents, pearlescent agents, opacifiers, sensates, suspending agents, solvents, diluents, anti-oxidants, vitamins, and mixtures thereof.

The personal cleansing composition may further comprise from about 0.01% to about 5%, preferably from about 0.1% to about 0.5% by weight of an antimicrobial agent, wherein the antimicrobial agent is selected from the group consisting of zinc pyrithione, climbazole, ketoconazole, itraconazole, econazole, elubiol, piroctone olamine, ciclopirox, rilopirox, MEA-Hydroxyoctyloxypyridinon, salicylic acid, strobilurins, azoxystrobin, 1,10-phenanthroline, and combinations thereof.

### Method

A method of increasing the rheology of a personal cleansing composition is provided and comprises the step of forming a personal cleansing composition as set out hereinbefore.

### Forms and Uses

### Product Form

The personal cleansing composition may be presented in typical personal cleansing formulations. They may be in the form of solutions, dispersion, emulsions, foams, and other delivery mechanisms. The personal cleansing composition may be a rinse-off composition.

The personal cleansing composition may be extrudable or dispensable from a single chamber package. The personal cleansing compositions can be in the form of liquid, semi-liquid, cream, lotion or gel, or solid compositions intended for topical application to skin.

Examples of personal cleansing compositions can include but are not limited to shampoo, conditioning shampoo, hair conditioner, body wash, moisturizing body wash, foaming body wash, shower gels, a shower or bath cream, skin cleansers, cleansing milks, hair & body wash, in shower body moisturizer, gel, emulsion, oil, mousse or spray.

The personal cleansing composition may be not in the form of a liquid hand wash or a liquid hand sanitizer.

The personal cleansing composition may be not a soap or a bar soap.

The personal cleansing composition may be a shampoo, wherein the shampoo comprises from about 0.01% to about 2%, preferably from about 0.1% to about 0.5% by weight of a cationic polymer, wherein the cationic polymer is selected from the group consisting of hydroxylpropyltrimonium guar, Polyquaternium-10, Polyquaternium-6 and combinations thereof, preferably Polyquaternium-10.

Suitable cationic cellulose polymers are salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Non-limiting examples include: JR-30M, KG-30M, JP, LR-30M, LR-400, JR-400 and mixtures thereof: KG30-M (MW 1.8 million g/mol, CD=1.9 meq/g JR30-M (MW-2.0 million g/mol, CD=1.25 meq/g), LR-30M (MW=1.8 million g/mol, CD=0.7 meq/g), LR-400 (MW=400,000 g/mol, CD=0.7 meq/g); JR-400 (MW=400,000 g/mol, CD=1.25 meq/g).

In that aspect, the shampoo may comprise lauramidopropyl betaine and/or cocamidopropyl betaine as the betaine.

The product forms contemplated for purposes of defining the personal cleansing compositions and methods are rinse-off formulations by which it is meant that the product is applied topically to the skin and then subsequently (i.e., within minutes) rinsed away with water, or otherwise wiped off using a substrate or other suitable removal means.

### Uses

The personal cleansing composition as set out hereinabove may be used for improving the lather of the composition.

The personal cleansing composition as set out hereinabove may be used for suspending benefits agents selected from the group consisting of hair care and skin care benefit agents, particulates, particles, preferably silica and titanium oxide, microcapsules, oils, droplets, pigments, opacifiers, pearlescent agents, feel modifiers, oil absorbers, skin protectants, matting agents, friction enhancers, slip agents, conditioning agents, exfoliants, odor absorbers, or cleaning enhancers, and mixtures thereof.

The personal cleansing composition can advantageously provide relatively improved ecotoxic or ecologically friendly environmental profile.

The personal cleansing composition can help to provide good esthetic properties such as good foam, and is thick and creamy in texture, is silky to the touch and affords conditioning.

### Test Methods

It is understood that the Test Methods that are disclosed in the Test Methods Section of the present application should be used to determine the respective values of the parameters described and claimed herein.

### Cone/Plate Viscosity Measurement

The viscosity of the personal cleansing composition is measured by a Cone/Plate Brookfield DV12T, by Brookfield Engineering Laboratories, Stoughton, MA. The cone used (Spindle CPA-41z) has a diameter of 24 mm and 3° angle. The viscosity is determined using a steady state flow experiment at constant shear rate of 2 s⁻¹ and at temperature of 26.5° C. The sample size is 2.5 mL.

### Product pH Measurement

First, calibrate the Metler TOLEDO PH meter. Do this by turning on the pH meter and waiting for 30 seconds. Then, take the electrode out of the storage solution, rinse the electrode with reversed osmosis (RO) water, and carefully wipe the electrode with a scientific cleaning wipe, such as a Kimwipe®. Submerse the electrode in the pH 4 buffer and press the calibrate button. Wait until the pH icon stops flashing. Rinse the electrode with RO water and carefully wipe the electrode with a scientific cleaning wipe. Then submerse the electrode into the pH 7 buffer and wait until the pH icon stops flashing. Rinse the electrode with RO water and carefully wipe with a scientific cleaning wipe. Then, submerse the electrode into the pH 9 buffer and wait until the pH icon stops flashing. Rinse the electrode with distilled RO water and carefully wipe with a scientific cleaning wipe. Now the pH meter is calibrated and can be used to test the pH of a solution.

### Fatty Acid Chain Length Distribution Test Method for an Acyl Isethionate Surfactant

Standard Solution—Prepare a known composition of lauric acid and tridecanoic acid separately with lauric acid as external standard solution and tridecanoic acid as internal standard solution. Transfer known amount of internal standard and external standard into a 20 mL vial and blow down to dryness with a nitrogen jet and then proceed with a derivatization and sampling steps as 1) add 2.4 mL of 0.5 N Methanolic Sodium Hydroxide Solution prepared by adding 2.0 grams sodium hydroxide into 100 mL methanol, shake to mix and heat the solution at 65° C. for 10 minutes then cool for at least 10 min to room temperature. 2) Add 3 mL of 12%-14% of boron trifluoride in methanol prepared by dissolving 12-14 g of boron trifluoride in methanol to make 100 mL, shake to mix, and heat the solution at 65° C. for 10 minutes then cool for at least 10 min to room temperature. 3) Add 2.4 mL of chromatographic n-heptane, and shake to mix, then add 9 mL of saturated sodium chloride solution to mix and allow the layers to separate. 4) Transfer the n-heptane layer into an auto-sampler vial for quantitation with Gas chromatograph.

Test Solution—Transfer about 110 mg of the pre-pulverized test specimen into a 20-mL volumetric flask with same amount of nitrogen dried internal standard as prepared in standard solution then proceed with a derivation and sampling steps as described in standard solution.

Chromatographic System—The gas chromatograph is equipped with a flame-ionization detector, maintained at a temperature of about 260° C., split-less injection system, and a 0.53-mm, 30-m fused-silica capillary column bonded with a 1.0-mm layer of USP phase G16. The chromatograph is programmed to maintain the column temperature at 70° C. for about 2 minutes after injection, then to increase the temperature at the rate of 5° C. per minute to 240° C., and finally to maintain this temperature for 10 minutes. The injection port temperature is maintained at about 220° C. The carrier gas is helium with a linear velocity of about 50 cm per second.

Chromatograph the System Suitability Solution, and record the peak responses as directed for Procedure: the resolution, R, between methyl stearate and methyl oleate is not less than 1.5; The fatty acid methyl esters are identified by commercially available fatty acid methyl ester mixture (Table 1) and should be resolved from other matrix peaks with minimum resolution of 1.2, for those matrix peaks could not be resolved from fatty acid methyl esters peaks (resolution <1.2), the matrix peak area should be less than 0.5% of corresponding fatty acid methyl esters peak area. The relative standard deviation of peak area ratio of methyl laurate to internal standard in the standard solution is no more than 1.0%. A known amount of standard solution would be injected as check sample, the recovery for check sample would be within range of 100±10.0%.

TABLE 1
A representative composition of fatty acid methyl ester mixture

| Fatty Acid Ester | Carbon-chain Length | No. of Double Bonds |
|---|---|---|
| Methyl caprylate | 8 | 0 |
| Methyl caprate | 10 | 0 |
| Methyl laurate | 12 | 0 |
| Methyl myristate | 14 | 0 |
| Methyl palmitate | 16 | 0 |
| Methyl stearate | 18 | 0 |
| Methyl oleate | 18 | 1 |
| Methyl linoleate | 18 | 2 |

Procedure—Separately inject equal volumes (about 1 mL) of the Standard Solution and the Test Solution into the chromatograph, record the chromatograms, identify the fatty acid ester peaks in the chromatogram of the Test Solution by comparing the retention times of these peaks with those obtained in the chromatogram of the fatty acid methyl ester mixture, and measure the peak areas for all fatty acid ester peaks in the chromatogram obtained from the Test Solution. Calculate the percentage of each fatty acid component of the test specimen in the sum of the peak areas of all the identified fatty acid ester peaks in the chromatogram obtained from the Test Solution.

### EXAMPLES

The following examples further describe the compositions described herein. The examples are given solely for the purpose of illustration and are not to be construed as limitations of the present disclosure, as many variations thereof are possible without departing from the spirit and scope of the disclosure. Where applicable, ingredients are identified by chemical or CTFA name, or otherwise defined below.

The following examples were prepared:
Compositions (Wt. %)

| Ingredients (wt. %) | C. Ex. 1 | C. Ex. 2 | C. Ex. 3 | C. Ex. 4 |
|---|---|---|---|---|
| Sodium cocoyl isethionate¹ | 2.00 | 2.00 | 2.00 | 2.50 |
| Sodium lauroyl sarcosinate² | 4.30 | 4.30 | 2.50 | 2.50 |
| Cocamidopropyl betaine³ | 7.75 | 7.75 | 9.00 | 9.00 |
| Sodium salicylate⁶ | 0.40 | 0.40 | 0.40 | 0.40 |
| Sodium benzoate⁷ | 0.45 | 0.45 | 0.45 | 0.45 |
| Citric acid powder⁸ | q.s. | q.s. | q.s. | q.s. |
| | pH 5.00 | pH 6.10 | pH 5.65 | pH 5.65 |
| Fragrance | 1.0 | 1.0 | 1.0 | 1.0 |
| Sodium chloride | — | — | 4.00 | 4.00 |
| water | Balance | Balance | Balance | Balance |
| | to | to | to | to |
| | 100 | 100 | 100 | 100 |
| Ratio betaine:sarcosinate | 1.80 | 1.80 | 3.60 | 3.60 |
| Ratio sarcosinate:isethionate | 2.15 | 2.15 | 1.25 | 1.00 |
| Initial viscosity (cPs) | 1031 | 50 | 1817 | 2591 |
| Viscosity after 3 weeks at | 610 | 120 | — | — |
| 60° C. (cPs) | | | | |
| Stability results | Phase | No | No | No |
| | sepa- | Sepa- | Sepa- | Sepa- |
| | ration | ration | ration | ration |

Table 2 depicts the chain length distributions of the acyl isethionate surfactants used. These description apply to the acyl isethionate surfactants used in the following examples.

TABLE 2
Acyl isethionate surfactant chain length distributions

| | C8 | C10 | C12 | C14 | C16 | C18 | C18:1 | C18:2 |
|---|---|---|---|---|---|---|---|---|
| Sodium acyl | 7.78% | 7.93% | 58.83% | 16.52% | 5.67% | 3.25% | 0.01% | 0.01% |
| isethionate 1 | | | | | | | | |
| (SCI 1) | | | | | | | | |
| Sodium acyl | 1.93% | 1.98% | 52.47% | 21.06% | 12.80% | 9.72% | 0.02% | 0.02% |
| isethionate 2 | | | | | | | | |
| (SCI 2) | | | | | | | | |
| Sodium acyl | 0.01% | 0.15% | 75.84% | 18.96% | 3.80% | 1.22% | 0.01% | 0.01% |
| isethionate 3 | | | | | | | | |
| (SCI 3) | | | | | | | | |

The following examples were prepared:
Compositions (Wt. %)

| Ingredients (wt. %) | C. Ex. A | Ex. 1 | Ex. 2 | C. Ex. B | Ex. 3 | Ex. 4 |
|---|---|---|---|---|---|---|
| Sodium acyl isethionate | 2.00 | — | — | 3.00 | — | — |
| SCI 1 | | | | | | |
| Sodium acyl isethionate | — | 2.00 | — | — | 3.00 | — |
| SCI 2 | | | | | | |
| Sodium acyl isethionate | — | | 2.00 | — | — | 3.00 |
| SCI 3 | | | | | | |
| Sodium lauroyl sarcosinate² | 2.00 | 2.00 | 2.00 | 4.00 | 4.00 | 4.00 |
| Cocamidopropyl betaine³ | 7.20 | 7.20 | 7.20 | 8.00 | 8.00 | 8.00 |
| Sodium salicylate⁶ | 0.40 | 0.40 | 0.40 | 0.40 | 0.40 | 0.40 |
| Sodium benzoate⁷ | 0.45 | 0.45 | 0.45 | 0.45 | 0.45 | 0.45 |
| Citric acid powder⁸ | q.s. | q.s. | q.s. | q.s. | q.s | q.s. |
| | pH 5.6 | pH 5.6 | pH 5.6 | pH 5.65 | pH 5.6 | pH 5.6 |
| Fragrance | 1.0 | 1.0 | 1.0 | 1.0 | 1.0 | 1.0 |
| Sodium chloride | 2.00 | 2.00 | 2.00 | 2.00 | 2.00 | 2.00 |
| water | Balance | Balance | Balance | Balance | Balance | Balance |
| | to 100 | to 100 | to 100 | to 100 | to 100 | to 100 |
| Ratio betaine:sarcosinate | 3.60 | 3.60 | 3.60 | 2.00 | 2.00 | 2.00 |
| Ratio sarcosinate:isethionate | 1.00 | 1.00 | 1.00 | 1.33 | 1.33 | 1.33 |
| Initial viscosity (cPs) | 1965 | 2947 | 8019 | 6398 | 9591 | 21230 |

### Definitions of Components

*1 Sodium cocoyl isethionate; Supplier Clariant as HOSTAPON SCI 85C
*2 Sodium lauroyl sarcosinate; Supplier Tinci
*3 Cocamidopropyl Betaine; Supplier Tinci
*6 Sodium salicylate; Supplier JQC Huayn Pharmaceutical Co Ltd.
*7 Sodium benzoate; Supplier Wuhan Youji Industries
*8 Citric acid powder; Supplier Yixing Union Biochemical
q.s.: sufficient quantity

### Results

C. Ex. 1 is a personal cleansing composition comprising sodium cocoyl isethionate, sodium lauroyl sarcosinate and cocamidopropyl betaine at a pH 5.0. After 3 weeks at 60° C., phase separation has been observed with a significant drop of the initial viscosity.

The top floating layer is a lamellar phase layer which is relatively highly ordered and comprises the above surfactants with relatively less water. As their density is lower, the lamellar phase floats and is the upper layer. The micellar phase is the lower layer.

The free fatty acids originating from sodium cocoyl isethionate and sodium lauroyl sarcosinate due to the respective hydrolysis of the anionic surfactants may contribute for the phase transition and phase separation from a micellar phase to a lamellar phase.

Initially, the hydrolysis of sodium cocoyl isethionate and sodium lauroyl sarcosinate causes the viscosity to increase due to the elongation of the wormlike micelles. After the generation of a certain amount of free fatty acid, however, the wormlike and spherical micelles transition from being wormlike micelles to bigger, more complex aggregates, such as disc-like micelles or gel networks. At that moment, these structures decrease the number of entanglements, reduce the resistance to flow, and increase fluidity of the system, resulting in a viscosity drop. A lamellar phase separated from a micellar phase.

When pH is increased from 5.0 to 6.1 in C. Ex. 2, the kinetics of the hydrolysis of sodium cocoyl isethionate and sodium lauroyl sarcosinate have been slowed down. No phase separation was observed. However, still the rheological properties of the personal cleansing composition was not satisfactory to be dispensed to the consumer.

C. Ex. 3 and C. Ex. 4 show that by adjusting the levels of sodium lauroyl sarcosinate, sodium cocoyl isethionate enhanced viscosity at 2.59 Pa·s (2591 cPs) could be reached.

Sodium cocoyl isethionate used in C. Ex. 1-4 are commercially available from Clariant as HOSTAPON SCI 85 C. In such material, the levels of each of C8 and C10 chains in the acyl isethionate surfactant are above 3.5 wt. %.

Several inventive and comparative composition were prepared and the rheology profile was measured for each according to the viscosity measurement method provided herein.

Now starting from C. Ex. 5 or C. Ex. 6 having sodium acyl isethionate (SCI 1) with a similar distribution as Clariant as HOSTAPON SCI 85 C, it has been found that the viscosity could be further enhanced when replacing sodium acyl isethionate (SCI 1) by sodium acyl isethionate (SCI 2) and further by sodium acyl isethionate (SCI 3) at the same level of 2 or 3 wt. % and at same constant level of electrolyte.

It has been found that a specific chain length distribution for the acyl isethionate surfactant could be provided to enhance the viscosity of the personal cleansing composition above 2 Pa·s (2000 cPs) or even above 8 Pa·s (8000 cPs) by not only lowering the proportion of C8 and C10 chains, but also by having a respective increase in the proportion of C12-C18, especially either by increasing the proportion of C12-C14 and/or C16-18.

The personal cleansing compositions do not include any polymeric rheology modifiers or increased levels of electrolyte to enhance the rheology profile and provide additional consumer benefits like lather and skin use feel.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal cleansing composition comprising:
a) a surfactant system, wherein the surfactant system comprises:
(i) from about 1.75% to about 15%, of an acyl isethionate surfactant by weight of the composition;
wherein a chain length distribution in the acyl isethionate surfactant is such that:
C8 chain within the chain length distribution is between about 0.01 wt. % to about 3.5 wt. %;
C10 chain within the chain length distribution is between about 0.01 wt. % to about 3.5 wt. %;
C12 chain within the chain length distribution is between about 40 wt. % to about 80 wt. %;
C14 chain within the chain length distribution is between about 10 wt. % to about 25 wt. %;
C16 chain within the chain length distribution is between about 3 wt. % to about 14 wt. %; and
C18 chain within the chain length distribution is between about 1 wt. % to about 14 wt. %;
b) wherein the pH is from about 5.5 to about 7; and
c) wherein the composition is substantially free of alkyl sulfate and alkyl ether sulfate type of surfactants.

2. The personal cleansing composition of claim 1 comprising:
wherein the surfactant system comprises:
(i) from about 1.75% to about 15%, of the acyl isethionate surfactant by weight of the composition;
(ii) from about 1.75% to about 10%, of a fatty acyl sarcosinate surfactant by weight of the composition;
(iii) from about 3% to about 20%, of a zwitterionic surfactant by weight of composition, wherein the zwitterionic surfactant comprises a betaine.

3. The personal cleansing composition of claim 2 comprising:
wherein the surfactant system comprises:
(i) from about 2.0% to about 3.0%, of the acyl isethionate surfactant by weight of the composition;
(ii) from about 1.75% to about 4.0%, of the fatty acyl sarcosinate surfactant by weight of the composition;
(iii) from about 7.2% to about 10%, of the betaine.

4. The personal cleansing composition of claim 1, wherein the weight ratio of betaine to fatty acyl sarcosinate surfactant is equal or above about 2.0:1, or from about 3.4:1 to about 3.8:1.

5. The personal cleansing composition of claim 1, wherein the weight ratio of fatty acyl sarcosinate surfactant to cocoyl isethionate is above about 0.5:1, or from about 0.65:1 to about 1.75:1.

6. The personal cleansing composition of claim 1, wherein the acyl isethionate surfactant is selected from the group consisting of sodium cocoyl isethionate, sodium cocoyl methyl isethionate, a blend of stearic acid and sodium cocoyl isethionate, ammonium cocoyl isethionate, ammonium cocoyl methyl isethionate, and mixtures thereof.

7. The personal cleansing composition of claim 1, wherein the fatty acyl sarcosinate surfactant is a sarcosinate according to the general formula (I): 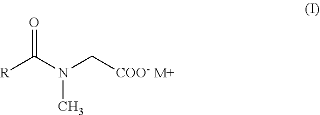
wherein R is a saturated or unsaturated, straight or branched or alkenyl, or alkyl chain with 7 to 17 carbon atoms, and M⁺ is H, a sodium, potassium or ammonium cation.

8. The personal cleansing composition of claim 7, wherein the fatty acyl sarcosinate surfactant is selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroyl glutamate/lauroyl sarcosinate, lauroyl sarcosinate, isopropyl lauroyl sarcosinate, potassium cocoyl sarcosinate, potassium lauroyl sarcosinate, sodium oleoyl sarcosinate, sodium palmitoyl sarcosinate, TEA-lauroyl sarcosinate, TEA-oleoyl sarcosinate, TEA-palm kernel sarcosinate, and mixtures thereof.

9. The personal cleansing composition of claim 1, wherein the betaine is selected from the group consisting of cocamidopropyl betaine, lauramidopropyl betaine, coco betaine, and mixtures thereof.

10. The personal cleansing composition of claim 9, wherein the composition comprises cocamidopropyl betaine and sodium lauroyl sarcosinate at a weight ratio betaine to sarcosinate equal or above 2:1; or from about 3.4:1 to about 3.8:1.

11. The personal cleansing composition of claim 9, wherein the composition comprises sodium lauroyl sarcosinate and sodium cocoyl isethionate at a weight ratio sarcosinate to isethionate from about 1:0.65 to about 1.75:1.

12. The personal cleansing composition of claim 1, wherein the personal cleansing composition further comprises from about 0.05% to about 5%, of an electrolyte by weight of the composition, wherein the electrolyte is selected from the group of sodium or potassium citrate, calcium chloride, calcium bromide, zinc chloride, barium chloride, calcium nitrate, potassium chloride, sodium chloride, potassium iodide, sodium bromide, ammonium bromide, sodium sulfate, and mixtures thereof.

13. The personal cleansing composition of claim 1, wherein the personal cleansing composition further comprises from about 0.01 wt. % to about 2 wt. % of a fragrance component by weight of the composition.

14. The personal cleansing composition of claim 1, wherein the composition comprises from about 0.01% to about 1.0%, of a preservative by weight of the composition; wherein the preservative includes a salicylate salt and a benzoate salt, wherein a total amount of the salicylate salt and the benzoate salt is from about 0.2% to about 0.9%, by weight of the composition.

15. The personal cleansing composition of claim 14, wherein the weight ratio of the salicylate salt to the benzoate salt is from about 1:1.10 to about 1:1.20.

16. The personal cleansing composition of claim 1, wherein the composition is selected from the group consisting of shampoo, conditioning shampoo, hair conditioner, body wash, moisturizing body wash, foaming body wash, shower gels, a shower or bath cream, skin cleansers, cleansing milks, hair and body wash, in shower body moisturizer.

17. The personal cleansing composition of claim 16, wherein the composition is a shampoo, wherein the shampoo comprises from about 0.01% to about 2% of a cationic polymer, wherein the cationic polymer is selected from the group consisting of hydroxylpropyltrimonium guar, Polyquaternium-10, Polyquaternium-6 and combinations thereof.

18. The personal cleansing composition of claim 1, wherein the composition further comprises from about 0.01% to about 5% of an antimicrobial agent, wherein the antimicrobial agent is selected from the group consisting of zinc pyrithione, climbazole, ketoconazole, itraconazole, econazole, elubiol, piroctone olamine, ciclopirox, rilopirox, MEA-Hydroxyoctyloxypyridinon, salicylic acid, strobilurins, azoxystrobin, 1,10-phenanthroline, and combinations thereof.

19. A method of increasing the rheology of a personal cleansing composition comprising the step of forming a personal cleansing composition of claim 1.
